(19) United States
(12) Patent Application Publication
(10) Pub. No.: US 2004/0102468 A1
(43) Pub. Date: May 27, 2004

(54) Utilization of buprenorphine in urinary incontinence therapy

(75) Inventors: Johannes Bartholomaeus, Aachen (DE); Thomas Christoph, Aachen (DE)

Correspondence Address: CROWELL & MORING LLP, INTELLECTUAL PROPERTY GROUP, P.O. BOX 14300, WASHINGTON, DC, 20044-4300, US

(73) Assignee: Gruenenthal GmbH, Aachen (DE)

(21) Appl. No.: 10/641,296

(22) Filed: Aug. 15, 2003

Related U.S. Application Data
(63) Continuation of application No. PCT/EP02/01699, filed on Feb. 18, 2002.

(30) Foreign Application Priority Data
Feb. 16, 2001 (DE) 101 07 828.5
Sep. 18, 2001 (DE) 201 15 429.3
Dec. 19, 2001 (DE) 101 62 704.1

(51) Int. Cl. A61K 31/485

(52) U.S. Cl. 514/282

(57) ABSTRACT

Methods for the use of buprenorphine compounds for treating increased urinary urgency, increased micturition, and/or urinary incontinence are disclosed, as well as corresponding medicaments and the production thereof.

### CROSS REFERENCE TO RELATED APPLICATIONS

This application is a continuation of International Patent Application No. PCT/EP02/01699, filed Feb. 18, 2002, designating the United States of America, and published in German as WO 02/066031, the entire disclosure of which is incorporated herein by reference. Priority is claimed based on the following Federal Republic of Germany patent applications: (1) Application No. DE 101 07 828.5, filed Feb. 16, 2001; (2) Application No. DE 201 15 429.3, filed Sep. 18, 2002; and (3) Application No. DE 101 62 704.1, filed Dec. 19, 2001.

### FIELD OF THE INVENTION

The invention relates to the use of buprenorphine for the preparation of a medicament for treatment of an increased urge to urinate, an increased frequency of micturition and/or urinary incontinence and to corresponding medicaments and methods for treatment of an increased urge to urinate, an increased frequency of micturition and/or urinary incontinence.

### BACKGROUND OF THE INVENTION

Urinary incontinence is the involuntary discharge of urine. This occurs in an uncontrolled manner when the pressure within the urinary bladder exceeds the pressure needed to close the ureter. Causes can be on the one hand an increased internal pressure in the bladder (e.g. due to detrusor instability) with the consequence of urgency incontinence, and on the other hand a reduced sphincter pressure (e.g. following giving birth or surgical interventions) with the consequence of stress incontinence. The detrusor is the coarsely bundled multilayered bladder wall musculature, contraction of which leads to voiding of urine, and the sphincter is the closing muscle complex of the urethra. Mixed forms of these types of incontinence and so-called overflow incontinence (e.g. in cases of benign prostate hyperplasia) or reflex incontinence (e.g. following damage to the spinal cord) occur. Further details in this respect are to be found in Chutka, D. S. and Takahashi, P. Y., 1998, Drugs 560: 587-595.

The urge to urinate is the state, aimed at voiding of urine (micturition), of increased bladder muscle tension as the bladder capacity is approached (or exceeded). The clinical picture of urgency incontinence here comprises 1. an increased urge to urinate, 2. an increased frequency of micturition and 3. involuntary urinary incontinence as such. Causes can be, inter alia, inflammations of the urinary bladder and neurogenic bladder disorders, and also bladder tuberculosis. However, not all the causes have yet been clarified. Another clinical picture which fits here is hyperactive bladder (overactive bladder).

An increased urge to urinate, an increased frequency of micturition and also urinary incontinence are perceived as extremely unpleasant and there is a clear need among persons affected by these indications to achieve an improvement which is as long-term as possible.

An increased urge to urinate, an increased frequency of micturition and in particular urinary incontinence are conventionally treated with medicaments using substances which are involved in the reflexes of the lower urinary tract (Wein, A. J., 1998, Urology 51 (suppl. 21): 43-47). These are usually medicaments which have an inhibiting action on the detrusor muscle, which is responsible for the internal pressure in the bladder. These medicaments are e.g. parasympatholytics, such as oxybutynin, propiverine or tolterodine, tricyclic antidepressants, such as imipramine, or muscle relaxants, such as flavoxate. Other medicaments, which in particular increase the resistance of the urethra or of the neck of the bladder, show affinities for α-adrenoreceptors, such as ephedrine, for β-adrenoreceptors, such clenbutarol, or are hormones, such as oestradiol. Certain diarylmethylpiperazines and -piperidines are also described for this indication in WO 93/15062. For tramadol also a positive effect on bladder function has been demonstrated in a rat model of rhythmic bladder contractions (Nippon-Shinyaku, WO 98/46216).

There is furthermore literature in which a known side effect of opiods, urinary retention, is investigated clinically (Cousins and Mather, 1984, Anesthesiol. 61, 276-310). Examples are weak opioids, such as diphenoxylate (Fowler et al., 1987 J. Urol 138:735-738), potent opioids, such as morphine (Malinovsky et al., 1998 loc. cit.; Kontani and Kawabata, (1988); Jpn J Pharmacol. Sep;48(1):31) and meperidine (Doyle and Briscoe, 1976 Br J Urol 48:329-335; Mohan et al., 1995, Int. J. Clin. Pharmacol. Therap. 33, 34-37) and very potent opioids, such as fentanyl (Malinovsky et al., 1998 loc. cit.; Drenger and Magora, 1989 Anesth Analg 69:348-353) or also mixed opioid agonists/antagonists, such as pentazocine (Shimizu et al. (2000) Br. J. Pharmacol. 131 (3): 610- 616; Mohan et al., 1995, Int. J. Clin. Pharmacol. Therap. 33, 34-37) and nalbuphine (Malinovsky et al., 1998, loc. cit.). Nevertheless, all these studies were carried out in analgesically active concentrations, since they were after all studies on humans, and in none of these cases has a positive effect ever been reported in the treatment of an increased urge to urinate or urinary incontinence. Rather, urinary retention is found here, which is, however, generally an entirely undesirable action and therefore makes these compounds appear unattractive.

In the case of the indications in question here, however, it should furthermore be remembered that it is in general a matter of very long-term uses of medicaments and, in contrast to many situations where analgesics are employed, those affected are faced with a situation which is very unpleasant but not intolerable. It is therefore to be ensured here—even more so than with analgesics—that side effects are avoided if the person affected does not want to exchange one evil for another. Also, analgesic actions are also largely undesirable during permanent treatment of urinary incontinence.

### SUMMARY OF INVENTION

The object of the present invention was therefore to provide substances which are helpful for treatment of an increased urge to urinate, an increased frequency of micturition or urinary incontinence and at the active doses preferably simultaneously show fewer side effects and/or less analgesic activity.

These and other object are achieved in accordance with the present invention by providing a method of treating a patient suffering from an increased urge to urinate, an increased frequency of micturition, urinary incontinence, urgency incontinence, or an overactive bladder, said method comprising administering to said patient a pharmaceutically effective amount of buprenorphine.

Surprisingly, it has now been found that buprenorphine already has a favourable action on bladder function, in particular urgency incontinence or “overactive bladder”, at low concentrations and is particularly suitable for treatment of corresponding clinical pictures.

The invention accordingly provides the use of buprenorphine, also in the form of its racemates, enantiomers and diastereomers, in particular in the form of mixtures of its enantiomers or diastereomers or in the form of an individual enantiomer or diastereomer; its base and/or salts of physiologically acceptable acids, for the preparation of a medicament for treatment of an increased urge to urinate, an increased frequency of micturition and/or urinary incontinence, in particular urgency incontinence or “overactive bladder”.

Surprisingly, it has been found that in a model with which the indications claimed, in particular urgency incontinence, can be simulated, buprenorphine is highly active. In the model, buprenorphines eliminates the detrusor overactivity induced by oxy-haemoglobin and correspondingly influences bladder parameters in a positive manner. Precisely in a model which clearly shows the disease symptoms just such as urgency incontinence, “overactive bladder” etc. buprenorphine has therefore proved suitable.

This action is also surprising in as much as there are also studies on buprenorphine in respect of urine retention and the action on bladder and urethra activity (Murray K., 1983, Brit. Med. J. 286, 765-766; Drenger and Magora, 1989 Anesth Analg 69:348-353: Batra et al., 1996, Int. J. Clin. Pharmacol. Therap. 34, 309-311; Malinovsky et al., 1998 Anesth Analg 87:456-461), wherein, however, the sometimes somewhat contradictory results contradict precisely a use of buprenorphine in urinary incontinence. Thus, Drenger and also Batra report that an epidural administration of buprenorphine in the analgesically active region of 4 μg/kg (Batra) or at 2 μg/kg (Drenger [on dogs]) has no significant influence on bladder or urethra function and in the end, due to a lack of effect, both therefore recommend precisely the use of buprenorphine for pain treatment on patients in whom complications in the urinary system are to be avoided. It is all the more astonishing that precisely buprenorphine shows a decidedly favourable action here in the treatment of urinary incontinence.

In the other extreme, Malinovsky reports (table 4, p. 460) that on administration of 0.3 mg i.v. to patients of about 70 kg body weight (4.3 μg/kg) urinary retention occurs in 5 out of 10 cases, and in the case described by Murray urinary retention also occurs after sublingual intake of 400 μg buprenorphine (˜5.7 μg/kg). Apart from the fact that urinary retention is a clearly undesirable action, the data here are contradictory and the doses reported in which adverse actions are shown on the bladder as a result cover or overlap with those in which none at all are shown. It is particularly confusing here that an amount of 4 μg/kg administered i.t. (Batra et al.) has no influence on bladder function in spite of the considerably lower distribution compartment and therefore higher concentration at the site of action, although precisely IT administration of opioids is said to lead to significant urinary retention (Cousins and Mather 1984; Drenger and Mangora 1989), while 4.3 μg/kg i.v. is said to lead to 50% urinary retention (Malinovsky et al.). It was all the more astonishing that in spite of the negative literature indications on both sides, buprenorphine is active on urinary incontinence, and in particular in a model which simulates the disease.

### DETAILED DESCRIPTION OF PREFERRED EMBODIMENTS

Suitable salts in the context of this invention and in each of the uses claimed are salts of buprenorphine with inorganic or organic acids and/or a sugar substitute, such as saccharin, cyclamate or acesulfam. The free base, the hydrochloride, stearate, citrate or lactate is particularly preferred here, in particular the free base or the hydrochloride.

It is particularly preferred if the treatment of an increased urge to urinate, an increased frequency of micturition and/or urinary incontinence with the medicaments prepared using, according to the invention, buprenorphine is carried out with a buprenorphine dose below the lower limit of the conventional dose for pain treatment.

It is very particularly preferred here if the treatment is carried out with an amount of buprenorphine of <300 μg or <4.3 μg/kg of body weight, preferably between 300 μg and 1 μg or 4.3 μg/kg and 0.014 μg/kg, in particular between 250 μg and 5 μg or 3.6 μg/kg and 0.07 μg/kg, particularly preferably between 200 μg and 10 μg or 2.8 μg/kg and 0.14 μg/kg. It is furthermore preferable that the stated amounts of buprenorphine are the maximum or minimum amounts of an individual dose and/or the maximum or minimum amounts administered per day.

Dosages (i.m. or i.v.) of 0.3-0.6 mg are stated as conventional in pain treatment (Martindale (ed. C. Parfitt), The complete drug reference, 32nd ed., 1999, p. 22 et seq.). Approx. 300 μg can accordingly be assumed here to be the lower limit of analgesic activity. The relative dosage in μ/kg was calculated accordingly for a patient with an average body weight of 70 kg.

In view of the data, for example of Batra et al. 1996, who saw no significant effects on the bladder with buprenorphine in humans with a dosage of 4 μg/kg, it was very surprising to discover that in spite of amounts of buprenorphine which already lie at this and furthermore also at significantly below this lower analgesic dosage, an action of buprenorphine which alleviates urgency incontinence arises in an animal model.

It is furthermore particularly preferred if the medicament prepared using, according to the invention, buprenorphine for treatment of an increased urge to urinate, an increased frequency of micturition and/or urinary incontinence shows a delayed release, and is preferably in the form of a sustained release formulation.

This is a very particularly preferred embodiment of the invention, since the treatment of urinary incontinence requires a very long-term treatment. It is therefore very favourable if the medicament shows a delayed release and the active compound is correspondingly released continuously over a relatively long period of time.

It is a preferred embodiment of the invention here, on the one hand, if the medicament is in the form of a delayed-release particle or implant, in particular an implant or particle of synthetic material, the synthetic material preferably being chosen from polylactide, polyglycollide or a polylactide/polyglycollide copolymer.

In this embodiment the buprenorphine is preferably bonded non-covalently to and in the particle or the implant, which, after administration, releases the active compound slowly, sometimes in the case of implants over months, and continuously in small amounts, usually with breakdown of the carrier matrix of the particle or implant. However, precisely because in the treatment of urgency incontinence symptoms, such as urinary incontinence, with buprenorphine only such surprisingly low doses are necessary and a continuously slow release is appropriate for treatment according to present knowledge, it has been found that this form of the invention is very favourable.

On the other hand, it is therefore also a very particularly preferred embodiment of the invention if the medicament prepared is a transdermal therapeutic system in the form of a patch for administration of buprenorphine to the skin. Also or precisely this form of the medicament prepared using, according to the invention, buprenorphine for treatment of an increased urge to urinate, an increased frequency of micturition and/or urinary incontinence shows, according to present knowledge, particularly favourable properties in this indication or these indications. In a favourable manner, such patches continuously release, over a period of 3 or also 5 or more days, particular easily adjustable amounts of buprenorphine, which can also be very low (which surprisingly are sufficient in this indication), which are then absorbed via the skin. Correspondingly suitable patches are known, inter alia, from EP 0 430 019 B1, WO 98/36728 or WO 96/19975.

It is preferred if the transdermal therapeutic system comprises a backing layer which is permeable to the active compound, an adhesive reservoir layer and a re-detachable protective layer. It is furthermore preferred here if the reservoir layer contains 20-90 wt. % of polymer material, 0.1-30 wt. % of plasticizer, 0.1-20 wt. % of buprenorphine, also in the form of its racemates, enantiomers or diastereomers, in particular in the form of mixtures of its enantiomers or diastereomers or in the form of an individual enantiomer or diastereomer; its base and/or salts of physiologically acceptable acids, preferably in the form of buprenorphine base, and 0.1-30 wt. % of a solvent for buprenorphine, the solvent for buprenorphine in the reservoir layer which remains in the system preferably being a compound with at least one acid group.

A particularly preferred form of the medicament prepared using, according to the invention, buprenorphine for treatment of an increased urge to urinate, an increased frequency of micturition and/or urinary incontinence shows a release rate of the buprenorphine of between 1 μg/h and 40 μg/h, preferably between 2 μg/h and 35 μg/h, in particular between 5 μg/h and 20 μg/h, preferably between 5 μg/h and 10 μg/h. These are—as has emerged—particularly favourable release rates for these indications.

It is a further preferred embodiment of the medicament prepared using, according to the invention, buprenorphine for treatment of an increased urge to urinate, an increased frequency of micturition and/or urinary incontinence in the form of a transdermal therapeutic system in the form of a patch for administration of buprenorphine to the skin if the transdermal therapeutic system has a release rate of the buprenorphine of the first order over a dosage interval of 72 h, so that a maximum plasma concentration of between 20 pg/ml and 1,052 pg/ml is achieved, and if, during the treatment, the transdermal therapeutic system remains on the skin of the patient for a further dosage interval of at least 2 days, during which the transdermal therapeutic system shows release kinetics of the buprenorphine of zero order, so that the patients experience analgesia during the additional dosage interval of at least two days.

It is furthermore preferred here that during the additional dosage interval of at least 2 days a relative average release rate of between 0.3 μg/h and 21 μg/h, preferably between 0.3 μg/h and 9 μg/h or between 13 μg/h and 21 μg/h, in particular between 0.3 μg/h and 0.6 μg/h, between 0.7 μg/h and 1 μg/h, between 2 μg/h and 4 μg/h, between 4 μg/h and 7 μg/h or between 5 μg/h and 9 μg/h is maintained.

This form is also preferred if the corresponding transdermal therapeutic system has a release rate of the buprenorphine of the first order over a dosage interval of 72 h, so that approx. 72 h after use of this transdermal therapeutic system an average plasma concentration of between 20 pg/ml and 1,052 pg/ml, preferably between 85 pg/ml and 263 pg/ml, in particular between 20 pg/ml and 66 pg/ml, between 42 pg/ml and 132 pg/ml, between 169 pg/ml and 526 pg/ml, between 254 pg/ml and 789 pg/ml or between 339 pg/ml and 1,052 pg/ml is achieved.

Corresponding transdermal therapeutic systems in which approx. 72 h after use of this transdermal therapeutic system an average plasma concentration of between 20 pg/ml and 1,052 pg/ml and during the additional dosage interval of at least 2 days a relative average release rate of between 0.3 μg/h and 9 μg/h is present, or an average plasma concentration of between 85 pg/ml and 263 pg/ml and a relative average release rate of between 13 μg/h and 21 μg/h or an average plasma concentration of between 0 pg/ml and 66 pg/ml and a relative average release rate of between 0.3 μg/h and 0.6 μg/h or an average plasma concentration of between 42 pg/ml and 132 pg/ml and a relative average release rate of between 0.7 μand 1 μg/h or an average plasma concentration of between 169 pg/ml and 526 pg/ml and a relative average release rate of between 2 μg/h and 4 μg/h or an average plasma concentration of between 254 pg/ml and 789 pg/ml and a relative average release rate of between 4 μg/h and 7 μg/h or an average plasma concentration of between 339 pg/ml and 1,052 pg/ml and a relative average release rate of between 5 μg/h and 9 μg/h, are also favourable.

This embodiment is furthermore preferred if the transdermal therapeutic system remains on the skin of the patient for at least 5 days.

Although buprenorphine in the use according to the invention shows only mild side effects to none at all, it may also be of advantage, for example to avoid certain forms of dependency, also to use morphine antagonists, in particular naloxone, naltrexone and/or levallorphan, in addition to these compounds.

The invention also relates to medicaments for the treatment of an increased urge to urinate, an increased frequency of micturition and/or urinary incontinence which comprise, as the active compound, at least buprenorphine, also in the form of its racemates, enantiomers and diastereomers, in particular in the form of mixtures of its enantiomers or diastereomers or in the form of an individual enantiomer or diastereomer; its bases and/or salts of physiologically tolerated acids, and optionally additives and/or auxiliary substances.

Generally, these medicaments according to the invention and also the medicaments described above prepared using, according to the invention, buprenorphine for treatment of an increased urge to urinate, an increased frequency of micturition and/or urinary incontinence on the one hand can comprise additives and/or auxiliary substances and on the other hand can be present in the most diverse known medicament forms.

Suitable additives and/or auxiliary substances in the context of this invention are all the substances known to the expert from the prior art for achieving pharmaceutical formulations. Auxiliary substances can be, for example, water, ethanol, 2-propanol, glycerol, ethylene glycol, propylene glycol, polyethylene glycol, polypropylene glycol, glucose, fructose, lactose, sucrose, dextrose, molasses, starch, modified starch, gelatine, sorbitol, inositol, mannitol, microcrystalline cellulose, methylcellulose, carboxymethylcellulose, cellulose acetate, shellac, cetyl alcohol, polyvinylpyrrolidone, paraffins, waxes, naturally occurring and synthetic gums, acacia gum, alginates, dextran, saturated and unsaturated fatty acids, stearic acid, magnesium stearate, zinc stearate, glyceryl stearate, sodium lauryl sulfate, edible oils, sesame oil, coconut oil, groundnut oil, soya bean oil, lecithin, sodium lactate, polyoxyethylene and -propylene fatty acid esters, sorbitan fatty acid esters, sorbic acid, benzoic acid, citric acid, ascorbic acid, tannic acid, sodium chloride, potassium chloride, magnesium chloride, calcium chloride, magnesium oxide, zinc oxide, silicon dioxide, titanium oxide, titanium dioxide, magnesium sulfate, zinc sulfate, calcium sulfate, potash, calcium phosphate, dicalcium phosphate, potassium bromide, potassium iodide, talc, kaolin, pectin, crospovidone, agar and bentonite.

The choice of these auxiliary substances and the amounts thereof to be employed depend on whether the medicament is to be administered orally, intravenously, intraperitoneally, intradermally, intramuscularly, intranasally, buccally or locally. Formulations in the form of tablets, chewable tablets, coated tablets, capsules, granules, drops, juices or syrups are suitable for oral administration, and solutions, suspensions, easily reconstitutable dry formulations and sprays are suitable for parenteral, topical and inhalatory administration. Suppositories for use in the rectum are a further possibility.

Buprenorphine can be released from certain formulation forms in a delayed manner. Examples are sustained release tablet forms, but in particular also the use of buprenorphine in a depot in dissolved form, a barrier film or a patch, optionally with the addition of agents which promote penetration of the skin, as suitable examples for suitable percutaneous administration forms and also delayed release particles or implants.

Examples of auxiliary substances and additives for oral administration forms include disintegrants, lubricants, binders, fillers, mould release agents, where appropriate solvents, flavouring substances, sugar, in particular carrier agents, diluents, dyestuffs, antioxidants etc. Waxes or fatty acid esters, inter alia, can be used or suppositories, and carrier substances, preservatives, suspension auxiliaries etc. can be used for compositions for parenteral administration.

A particularly preferred form of the medicament according to the invention exists if the medicament shows a delayed release, preferably is present in the form of a sustained release formulation, in particular is present in the form of a delayed release particle or implant, preferably an implant or particle of a synthetic material, the synthetic material preferably being chosen from polylactide or a polylactide/polyglycollide copolymer or is a transdermal therapeutic system in the form of patch for administration of buprenorphine to the skin.

For the medicament according to the invention, the same preferred embodiments as have already been described above for medicaments prepared using, according to the invention, buprenorphine for treatment of an increased urge to urinate, an increased frequency of micturition and/or urinary incontinence otherwise apply.

The medicaments and pharmaceutical compositions according to the invention can be prepared with the aid of means, devices, methods and processes which are well-known in the prior art of pharmaceutical formulation, such as are described, for example, in “Remington's Pharmaceutical Sciences”, eds. A. R. Gennaro, 17th ed., Mack Publishing Company, Easton, Pa. (1985), in particular in part 8, chapter 76 to 93. However, other types of preparation, in particular for modern medicament forms, are also conceivable and known.

The invention furthermore also relates to a method for treatment of an increased urge to urinate, an increased frequency of micturition or urinary incontinence in which buprenorphine, in the form of its racemates, enantiomers or diastereomers, in particular in the form of mixtures of its enantiomers or diastereomers or in the form of an individual enantiomer or diastereomer; its bases and/or salts of physiologically acceptable acids, is used.

The following examples are intended to illustrate the invention without the subject matter of the invention being limited thereto.

### EXAMPLES

### Example 1

### Test System of Cystometry on Conscious Naive Rats

Cystometric studies were carried out on naive, female Sprague-Dawley rats by the method of Ishizuka et al. ((1997), Naunyn-Schmiedeberg's Arch. Pharmacol. 355: 787-793). Three days after implantation of bladder and venous catheters, the animals were examined in the conscious state, freely mobile. The bladder catheter was connected to a pressure transducer and an injection pump. The animals were placed in metabolism cages which allowed measurement of the volume of urine. Physiological saline solution was infused into the emptied bladder (10 ml/h) and the bladder pressure and micturition volume were recorded continuously. After a stabilization phase, a 20-minute phase which was characterized by normal, reproducible micturition cycles was recorded. The following parameters, inter alia, were determined:

Threshold pressure (TP, bladder pressure immediately before micturition),

Bladder capacity (BC, residual volume after preceding micturition plus volume of the infused solution during the filling phase),

Intercontraction interval ((ICI), the interval of time between micturitions).

An increase in the threshold pressure (TP) indicates an important therapeutic action on one of the indications according to the invention. The intercontraction interval (ICI) is also an important parameter for measuring the physiological activity of a substance in the treatment of urinary incontinence, as is the bladder capacity (BC). On the basis of the very heterogeneous causes of the symptoms of these clinical pictures, for an activity it is not necessary to have a positive influence on all three parameters here. It is therefore entirely sufficient if a positive action is to be found in only one of these parameters for it to be possible to employ the substance in urinary incontinence, an increased frequency of micturition or an increased urge to urinate.

After three reproducible micturition cycles were recorded as the pre-value, 10 μg/kg buprenorphine in the vehicle=0.9% NaCl were administered i.v. and the action on the cystometric parameters was recorded for 90 to 120 minutes. At the action maximum, the mean of 3 micturition cycles was determined and was stated as the percentage change with respect to the pre-value (table 1).

The concentration employed corresponds to the ED₅₀ in a known analgesia model for rats, the tail flick.

| | TP | | ICI |
|---|---|---|---|
| | threshold | BC | intercontraction |
| Buprenorphine | pressure | bladder capacity | interval |
| 0.01 mg/kg iv | +69.9% | +3.6% | +10.9% |
| (n = 6) | ** | | |

Table 1: Influence on the cystometric parameters by buprenorphine (change from the pre-value [%]); n corresponds to the number of animals employed in the study. Significance (Student T test): *p<0.05; ** p<.0.01; *** p<0.001.

Precisely on the TP buprenorphine shows a positive action on bladder regulation and is therefore suitable in principle for treatment of urinary incontinence. Nevertheless, the concentration employed, which has an analgesic action, is evidently too high, since drip incontinence occurred in 2 of the 6 animals. At two lower concentrations, 0.001 mg/kg i.v. and 0.005 mg/kg i.v., an increase in the TP of +27.6% and +37.5% respectively occurred at n=6.

### Example 2

### Test System of Cystometry on Conscious Damaged Rats

This model simulates urgency incontinence in an animal model; the oxyhaemoglobin (OxyHb) employed induces bladder overactivity.

Cystometric studies were carried out on naive, female Sprague-Dawley rats by the method of Pandita et al. (J. Urol. 2000, 164:545-550). Three days after implantation of bladder and venous catheters, the animals were examined in the conscious state, freely mobile. The bladder catheter was connected to a pressure transducer and an injection pump. The animals were placed in metabolism cages which allowed measurement of the volume of urine. Physiological saline solution was infused into the emptied bladder (10 ml/h) and the bladder pressure and micturition volume were recorded continuously. After a stabilization phase, a 20-minute phase which was characterized by normal, reproducible micturition cycles was recorded. The following parameters, inter alia, were determined:

Threshold pressure (TP, bladder pressure immediately before micturition),

Bladder capacity (BC, residual volume after preceding micturition plus volume of the infused solution during the filling phase),

Intercontraction interval ((ICI), the interval of time between micturitions)

Micturition pressure (MP, maximum bladder pressure during micturition).

An increase in the threshold pressure (TP) indicates an important therapeutic action on one of the indications according to the invention. The intercontraction interval (ICI) is also an important parameter for measuring the physiological activity of a substance in the treatment of urinary incontinence, as is the bladder capacity (BC). On the basis of the very heterogeneous causes of the symptoms of these clinical pictures, for an activity it is not necessary to have a positive influence on all the parameters here. It is therefore entirely sufficient if a positive action is to be found in only one of these parameters for it to be possible to employ the substance in urinary incontinence, an increased frequency of micturition or an increased urge to urinate.

After three reproducible micturition cycles were recorded as the pre-value, 2.5×10⁻⁴ M oxyhaemoglobin in the vehicle=0.9% NaCl were infused into the bladder. The action on the cystometric parameters were recorded for about 20 minutes. At the action maximum, the mean of 3 micturition cycles was determined and was stated as the percentage change with respect to the pre-value (table 2). Treatment with oxyhaemoglobin induces a characteristic change in the cystometric parameters with an increase in the micturition pressure, a lowering of the bladder capacity and a reduction in the intercontraction interval. These changes represent the changes found in patients with urgency incontinence.

Administration of 5 μg/kg buprenorphine in the vehicle=0.9% NaCl i.v. before administration of oxyhaemoglobin is capable of suppressing the changes induced by oxyhaemoglobin and moreover of also inducing an increase in the threshold pressure (table 2).

| | | | | ICI |
|---|---|---|---|---|
| | MP | TP | BC | inter- |
| | Micturition | threshold | bladder | contraction |
| | pressure | pressure | capacity | interval |
| | [cm H₂O] | [cm H₂O] | [ml] | [min] |
| OxyHb | | | | |
| 2.5 × 10⁻⁴ M iv | b: 59 ± 8 | b: 8.72 ± 1.31 | b: 0.92 ± 0.10 | b: 4.96 ± 0.33 |
| | a: 97 ± 5 | a: 9.84 ± 1.56 | a: 0.65 ± 0.06 | a: 3.33 ± 0.18 |
| (n = 5) | dif.: +64.4% | dif.: +12.8% | dif.: −29.3% | dif.: −32.9% |
| | ** | | ** | ** |
| OxyHb + | | | | |
| Buprenorphine | | | | |
| OxyHb: | b: 54 ± 9 | b: 9.07 ± 1.29 | b: 1.19 ± 0.12 | b: 6.72 ± 0.73 |
| 2.5 × 10⁻⁴ M | a: 37 ± 8 | a: 14.28 ± 2.53 | a: 1.17 ± 0.13 | a: 6.70 ± 0.88 |
| buprenorphine: | dif.: −31.5% | dif.: +57.4% | dif.: −1.7% | dif.: −0.3% |
| 0.005 mg/kg iv | * | * | | |
| (n = 6) | | | | |

Table 2: Influence on cystometric parameters by oxyhaemoglobin (OxyHb) with and without prior administration of buprenorphine. Average values with standard deviations before (b) and after (a) use of the substances and the change (dif.) compared with the pre-value [%] are stated; n corresponds to the number of animals employed in the study. Significance (Student T test):* p<0.05; ** p<0.01; *** p<0.001.

It can be seen that OxyHb has a significant adverse influence on bladder parameters in the sense of urgency incontinence. This adverse influence is eliminated and even improved by buprenorphine. The micturition pressure thus falls compared with the urgency incontinence induced by OxyHb, and even significantly compared with the untreated control. Furthermore, in this urgency incontinence model buprenorphine completely normalizes the intercontraction interval and the bladder capacity and also has the effect of a significant and clear increase in the threshold pressure.

The evidence is thus provided that buprenorphine, in particular in the field of urgency incontinence, for which the OxyHb model is a standard model, shows an outstanding action, and in particular also in the event of damage, that is to say in the case of disease.

### Example 3

### Transdermal Formulation

A transdermal administration system is formulated in accordance with example 1 of WO 98/36728.

1.139 g of a polyacrylate solution of 47.83 wt. % with self-crosslinking acrylate copolymers comprising 2-ethyl acrylate, vinyl acetate, acrylic acid (solvent: ethyl acetate : heptane : isopropanol : toluene : acetylacetonate in a ratio of 37:26:26:4:1), 100 g laevulinic acid, 150 g oleyl oleate, 100 g polyvinylpyrrolidone, 150 g ethanol, 200 g ethyl acetate and 100 g buprenorphine base were homogenised. The mixture was stirred for approx. 2 h and then inspected visually to determine whether all the solid substances had dissolved. Evaporation losses must be checked by renewed weighing and if necessary the solvent must be compensated with the aid of ethyl acetate. Thereafter, the mixture is applied to a transparent polyester film 420 mm wide such that the weight per unit surface area of the dried layer is 80 g/m². The polyester film serves as a protective layer and can be detached again by treatment with silicone. The solvent is removed by heated air passed over a damp zone. By this heat treatment, not only do the solvents evaporate, the laevulinic acid also melts. Thereafter, the sealing film is covered with a polyester film 15 μ thick. An area of 16 cm² is cut out with the aid of a suitable cutting tool and the edges which have remained between the individual objects are removed.

To achieve the nominal release rate of approx. 25 μg/h, the total amount of buprenorphine in the transdermal patch is approx. 10 mg, the active surface area are approx. 12.5 Cm² and the size of the patch is, for example, approx. 30.6 cm².

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof

What is claimed is:

1. A method of using treating a patient suffering from an increased urge to urinate, an increased frequency of micturition, urinary incontinence, urgency incontinence, or an overactive bladder, said method comprising administering to said patient a pharmaceutically effective amount of buprenorphine.

2. The method of claim 1, wherein said buprenorphine is administered in the form of an enantiomer, a diastereoisomer, a mixture of enantiomers or diastereoisomers.

3. The method of claim 1, wherein said buprenorphine is administered in the form of a racemic mixture.

4. The method of claim 1, wherein said pharmaceutically effective amount of buprenorphine is less than 300 μg.

5. The method of claim 1, wherein said pharmaceutically effective amount of buprenorphine comprises a buprenorphine dose of between 1 μg and 300 μg.

6. The method of claim 5, wherein said pharmaceutically effective amount of buprenorphine comprises a buprenorphine dose of between 5 μg and 250 μg.

7. The method of claim 6, wherein said pharmaceutically effective amount of buprenorphine comprises a buprenorphine dose of between 10 μg and 200 μg.

8. The method of claim 1, wherein said pharmaceutically effective amount of buprenorphine is less than 4.3 μg per kg of patient body weight.

9. The method of claim 1, wherein said pharmaceutically effective amount of buprenorphine comprises a buprenorphine dose of between 0.014 μg and 4.3 μg per kg of patient body weight.

10. The method of claim 9, wherein said effective amount of buprenorphine comprises a buprenorphine dose of between 0.07 μg and 3.6 μg per kg of patient body weight.

11. The method of claim 10, wherein said effective amount of buprenorphine comprises a buprenorphine dose of between 0.14 μg and 2.8 μg per kg of patient body weight.

12. The method of claim 1, wherein said buprenorphine is administered in a delivery form selected from the group consisting of sustained release formulations, delayed-release particles, implants, and transdermal therapeutic systems.

13. The method of claim 12, wherein said delivery form comprises a synthetic material selected from the group consisting of polylactide polymers, polyglycollide polymers, and polylactide/polyglycollide copolymers.

14. The method of claim 12, wherein the delivery form is a transdermal therapeutic system, and said transdermal therapeutic system remains on the skin of a patient for at least 5 days.

15. The method of claim 12, wherein said delivery form is a transdermal therapeutic system comprising:
a backing layer which is permeable to active compounds;
an adhesive reservoir layer; and
a re-detachable protective layer.

16. The method of claim 15, wherein said reservoir layer comprises:
20-90 weight percent of polymer material;
0.1-30 weight percent of plasticizer; and
0.1-20 weight percent of buprenorphine.

17. The method of claim 15, wherein said reservoir layer comprises 0.1 to 30 weight percent of a solvent for buprenorphine, the solvent for buprenorphine being in the reservoir layer and remaining in said transdermal therapeutic system.

18. The method of claim 17, wherein said solvent comprises at least one acid group.

19. The method of claim 12, wherein said delivery form is a transdermal therapeutic system, and said administering comprises:
applying the transdermal therapeutic system to the skin of a patient for a first dosage interval of 72 hours wherein the transdermal therapeutic system has a release rate of the buprenorphine of the first order such that a maximum plasma concentration of between 20 pg/ml and 1,052 pg/ml is achieved; and
applying the transdermal therapeutic system to the skin of the patient for a second dosage interval of at least 24 hours, during which second dosage period the transdermal therapeutic system has a release rate of the buprenorphine of zero order, such that the patient experiences analgesia during the second dosage interval.

20. The method of claim 19, wherein the transdermal therapeutic system has a relative average release rate of the buprenorphine of between 0.3 μg/hour and 21 μg/hour during the second dosage interval.

21. The method of claim 19, wherein the transdermal therapeutic system has a relative average release rate of the buprenorphine of between 0.3 μg/hour and 9 μg/hour during the second dosage interval.

22. The method of claim 19, wherein the transdermal therapeutic system has a relative average release rate of the buprenorphine of between 13 μg/hour and 21 μg/hour during the second dosage interval.

23. The method of claim 19, wherein the transdermal therapeutic system has a relative average release rate of the buprenorphine of between 0.3 μg/hour and 0.6 μg/hour during the second dosage interval.

24. The method of claim 19, wherein the transdermal therapeutic system has a relative average release rate of the buprenorphine of between 0.7 μg/hour and 1 μg/hour during the second dosage interval.

25. The method of claim 19, wherein the transdermal therapeutic system has a relative average release rate of the buprenorphine of between 2 μg/hour and 4 μg/hour during the second dosage interval.

26. The method of claim 19, wherein the transdermal therapeutic system has a relative average release rate of the buprenorphine of between 4 μg/hour and 7 μg/hour during the second dosage interval.

27. The method of claim 19, wherein the transdermal therapeutic system has a relative average release rate of the buprenorphine of between 5 μg/hour and 9 μg/hour during the second dosage interval.

28. The method of claim 19, wherein the transdermal therapeutic system has a relative average release rate of the buprenorphine of the first order over the first dosage interval of 72 hours, such that 72 hours after use of the transdermal therapeutic system an average plasma concentration of between 20 pg/ml and 1,052 pg/ml is achieved.

29. The method of claim 19, wherein the transdermal therapeutic system has a relative average release rate of the buprenorphine of the first order over the first dosage interval of 72 hours, such that 72 hours after use of the transdermal therapeutic system an average plasma concentration of between 85 pg/ml and 263 pg/ml is achieved.

30. The method of claim 19, wherein the transdermal therapeutic system has a relative average release rate of the buprenorphine of the first order over the first dosage interval of 72 hours, such that 72 hours after use of the transdermal therapeutic system an average plasma concentration of between 20 pg/ml and 66 pg/ml is achieved.

31. The method of claim 11, wherein the transdermal therapeutic system has a relative average release rate of the buprenorphine of the first order over the first dosage interval of 72 hours, such that 72 hours after use of the transdermal therapeutic system an average plasma concentration of between 42 pg/ml and 132 pg/ml is achieved.

32. The method of claim 19, wherein the transdermal therapeutic system has a relative average release rate of the buprenorphine of the first order over the first dosage interval of 72 hours, such that 72 hours after use of the transdermal therapeutic system an average plasma concentration of between 169 pg/ml and 526 pg/ml is achieved.

33. The method of claim 19, wherein the transdermal therapeutic system has a relative average release rate of the buprenorphine of the first order over the first dosage interval of 72 hours, such that 72 hours after use of the transdermal therapeutic system an average plasma concentration of between 254 pg/ml and 789 pg/ml is achieved.

34. The method of claim 19, wherein the transdermal therapeutic system has a relative average release rate of the buprenorphine of the first order over the first dosage interval of 72 hours, such that 72 hours after use of the transdermal therapeutic system an average plasma concentration of between 339 pg/ml and 1,052 pg/ml is achieved.

35. The method of claim 12, wherein said delivery form is a patch for administration of buprenorphine to the skin.

36. The method of claim 12, wherein said delivery form releases the buprenorphine at a rate between 1 μg/hour and 40 μg/hour.

37. The method of claim 36, wherein said delivery form releases the buprenorphine at a rate between 2 μg/hour and 35 μg/hour.

38. The method of claim 37, wherein said delivery form releases the buprenorphine at a rate between 5 μg/hour and 20 μg/hour.

39. The method of claim 38, wherein said dosage form releases the buprenorphine at a rate between 5 μg/hour and 10 μg/hour.

40. The method of claim 1, further comprising administering a morphine antagonist to said patient.

41. The method of claim 40, wherein said morphine antagonist is selected from the group consisting of naloxone, naltrexone, and levallorphan.

42. The method of claim 1, wherein said buprenorphine is a free base, a hydrochloride, a stearate, a citrate, or a lactate.

43. A pharmaceutical composition for treating a patient suffering from an increased urge to urinate, an increased frequency of micturition, urinary incontinence, urgency incontinence, or an overactive bladder comprising buprenorphine and at least one pharmaceutical carrier or auxiliary.

44. The pharmaceutical composition of claim 43, wherein said buprenorphine is present in the form of an enantiomer, a diastereoisomer, or a mixture of enantiomers or diastereoisomers.

45. The pharmaceutical composition of claim 43 wherein said buprenorphine is present in a delayed release or sustained release formulation.

46. The pharmaceutical composition of claim 45, wherein said buprrenorphine is present in form of a delayed release particle or implant.

47. The pharmaceutical composition of claim 46, wherein said delayed release particle or implant comprises a synthetic material selected from the group consisting of polylactide polymers, polyglycollide polymers, and polylactide/polyglycollide copolymers.

48. The pharmaceutical composition of claim 43, in the form of a transdermal patch for administration of buprenorphine to the skin.

49. The pharmaceutical composition of claim 43, wherein said buprenorphine is present as a salt of a physiologically acceptable acid.

50. The pharmaceutical composition of claim 43, wherein said buprenorphine is present as a free base.
